(19) United States
(12) Patent Application Publication
(10) Pub. No.: US 2019/0076491 A1
(43) Pub. Date: Mar. 14, 2019

(54) METHODS OF TREATING SUBSTANCE ABUSE RELATED DISEASES OR DISORDERS

(71) Applicant: PEACHTREE HEALTH MANAGEMENT LLC DBA AEON CLINICAL LABORATORIES, Gainsville, GA (US)

(72) Inventor: Shawn Desai, Gainsville, GA (US)

(73) Assignee: PEACHTREE HEALTH MANAGEMENT LLC DBA AEON LABORATORIES, Gainsville, GA (US)

(21) Appl. No.: 16/083,646

(22) PCT Filed: Mar. 6, 2017
(86) PCT No.: PCT/US17/20894

§ 371 Date: Sep. 10, 2018

Related U.S. Application Data
(60) Provisional application No. 62/306,367, filed on Mar. 10, 2016.

(51) Int. Cl. A61K 35/747; A61K 9/00; A61K 35/37; A23L 33/135

(52) U.S. Cl. CPC A61K 35/747; A61K 9/0053; A23L 33/135; A61K 9/0031; A61K 35/37

(57) ABSTRACT

Compositions and methods for treating a subject for a substance-abuse related disease or disorder are disclosed. The substance-abuse related disease or disorder can be an alcohol-related, an opioid-related, a cocaine-related, an amphetamine-related, a marijuana related, hallucinogen-related, barbiturate-related, benzodiazepine-related, or VOC-related disease or disorder. The composition for treating the substance-abuse related disease or disorder can be selected from a microbiota transplant, a particular diet or diet ingredient, a probiotic, or a combination thereof. In some embodiments, the composition can be a fecal transplant. The fecal transplant can be administered in a therapeutically effective amount to increase the amount of gastrointestinal microbiota when consumed by the subject. The methods described herein can further include administering a therapeutic agent in combination with the composition that modulates the gut microbiota.

### FIELD

The subject matter disclosed herein generally relates to compositions and methods for treating substance-abuse related diseases or disorders, particularly to using microbiota transplant for treating the substance-abuse related diseases or disorders.

### BACKGROUND

Substances that are often the basis of abuse and dependence include stimulants (amphetamine, methamphetamine, MDMA), opioids (morphine, heroin), barbiturates, hallucinogens (LSD), cocaine, hemp (marijuana), benzodiazepines (sedatives, hypnotics, anxiolytics), alcohol, and volatile organic solvents. Among these, alcohol use disorders and dependence are the most widespread. It is estimated that 16.6 million American adults abused alcohol or were dependent on it in 2013 and that approximately 10% of Americans will be affected by alcohol dependence sometime during their lives. Alcohol use disorders, characterized by the preoccupation with alcohol use, tolerance, and withdrawal, is a chronic disorder with genetic, psychosocial, and environmental factors influencing its development and manifestations. Studies have demonstrated the significance of opioids (i.e., beta-endorphin), dopamine (DA), serotonin (5-HT), γ-amino-butyric acid (GABA), and glutamate for the development and maintenance of alcohol dependence.

Various medications and behavioral therapy have been used to treat alcohol dependence. The neuronal targets of alcohol include many neurotransmitter systems and the molecules participating in or regulating the systems, including GABA, glutamate, DA, opioids, and serotonin (Johnson, 2004, Expert Opin. Pharmacother., 5:9:1943-1955). Despite the number of studies performed in this area, few drugs for alcohol dependence are approved in the U.S. The approved drugs are disulfiram, naltrexone, and acamprosate. Disulfiram is an irreversible inhibitor of aldehyde dehydrogenase leading to increased levels of acetaldehyde, a toxic intermediate in alcohol metabolism. Patients who take disulfiram and drink alcohol experience an increased dilation of arterial and capillary tone producing hypotension, nausea, vomiting, flushing, headache and possibly in some, worse symptoms. Therefore, the concept behind the use of disulfiram is that the alcohol-dependent individual associates drinking with unpleasant adverse events and, as a result, avoids further alcohol consumption. Nevertheless, recent research shows that disulfiram has limited utility because compliance is low unless it is administered by a partner or spouse.

While less widespread, opioid use disorders, such as addiction to heroin or prescription pain medications, are also a significant health concern. Methadone maintenance treatment for opioid dependence reduces morbidity, mortality, and the spread of infectious diseases but is restricted to licensed specialty clinics in the United States, requires frequent clinic visits, and has a high risk of overdose and dependence. Buprenorphine has also been used as a treatment for opioid addiction, and numerous studies support the efficacy of sublingually administered buprenorphine. However, there are several concerns about diversion and nonmedical use of sublingual buprenorphine. Poor treatment adherence, resulting in craving and withdrawal symptoms that increase the likelihood of relapse, is also a concern with sublingual buprenorphine.

Amphetamines, cocaine, barbiturates, hallucinogens (LSD), hemp (marijuana), benzodiazepines, and volatile organic solvents (VOC) are also the cause of abuse-related diseases and disorders. Abusers of such substances cannot stop because stopping causes withdrawal symptoms, making normal life without drugs impossible, rendering the person physically and mentally debilitated. What are needed are new compositions and methods for treating substance-abuse related diseases or disorders. The compositions and methods disclosed herein address these needs.

### SUMMARY

Compositions and methods for treating a subject for substance-abuse related diseases or disorders are disclosed herein. In some embodiments, the disease or disorder is an alcohol-related disease or disorder such as alcohol abuse, addiction, or dependency. In other embodiments, the disease or disorder is an opioid-related, a cocaine-related, an amphetamine-related, a marijuana-related, hallucinogen-related, barbiturate-related, benzodiazepine-related, or VOC-related disease or disorder. In still further embodiments, the disease or disorder is an amphetamine-related disease or disorder.

The compositions for treating the substance-abuse related diseases or disorders can modulate the gut microbiota in the subject. For example, the composition can be selected from a microbiota transplant, a particular diet or diet ingredient, a probiotic, or a combination thereof. In certain embodiments, the composition can include at least one bacterial species of a genera of Akkermansia, Ruminococcus, Pseudobutyrivibrio, Coprococcus, Coprobacillus, Lactobacillus, Bifidobacteria, Clostridia, Verrucomicrobiae, Verrucomicrobia, or Verrucomicrobiales. In certain embodiments, the composition can include an unknown and/or unculturable bacteria.

In some embodiments, the composition can be a fecal transplant. The fecal transplant can be administered in a therapeutically effective amount to increase the amount of gastrointestinal microbiota when consumed by the subject. In some embodiments, the fecal transplant comprises from about 10³ to about 10¹¹ colony forming units per gram of the composition. The fecal transplant can be administered by any suitable means, such as orally or anally.

The compositions disclosed herein can be administered intermittently, periodically, continuously, or chronically. In some embodiments, the compositions can be administered as a single dose. In some embodiments, the compositions can be administered once daily. In some embodiments, the compositions can be administered for up to three weeks.

The methods described herein can further include administering a therapeutic agent in combination with the composition that modulates the gut microbiota. The therapeutic agent can be selected from naltrexone, disulfiram, and acamprosate.

### DETAILED DESCRIPTION

The compositions and methods described herein may be understood more readily by reference to the following detailed description of specific aspects of the disclosed subject matter.

Before the present compositions and methods are disclosed and described in detail, it is to be understood that the aspects described below are not limited to specific microbiota composition, as such may, of course, vary. It is also to be understood that the terminology used herein is for the purpose of describing particular aspects only and is not intended to be limiting.

Also, throughout this specification, various publications are referenced. The disclosures of these publications in their entireties are hereby incorporated by reference into this application in order to more fully describe the state of the art to which the disclosed matter pertains. The references disclosed are also individually and specifically incorporated by reference herein for the material contained in them that is discussed in the sentence in which the reference is relied upon.

### General Definitions

In this specification and in the claims that follow, reference will be made to a number of terms, which shall be defined to have the following meanings:

Throughout the description and claims of this specification the word “comprise” and other forms of the word, such as “comprising” and “comprises,” means including but not limited to, and is not intended to exclude, for example, other additives, components, integers, or steps.

As used in the description and the appended claims, the singular forms “a,” “an,” and “the” include plural referents unless the context clearly dictates otherwise. Thus, for example, reference to “a composition” includes mixtures of two or more such compositions, reference to “the transplant” includes mixtures of two or more such transplants, and the like.

“Optional” or “optionally” means that the subsequently described event or circumstance can or cannot occur, and that the description includes instances where the event or circumstance occurs and instances where it does not.

The term “subject” refers to any individual who is the target of administration or treatment. The subject can be a vertebrate, for example, a mammal. Thus, the subject can be a human or veterinary patient. In some embodiments, the subject is a human. The term “patient” refers to a subject under the treatment of a clinician, e.g., physician.

The term “substance abuse” as used herein, refers to a maladaptive pattern of substance use leading to clinically significant impairment or distress, as manifested by one (or more) of the following, occurring over a period of time: (1) recurrent substance use resulting in a failure to fulfill major role obligations at work, school, or home; (2) recurrent substance use in situations in which it is physically hazardous; (3) recurrent substance-abuse related legal problems; and (4) continued substance use despite having persistent or recurrent social or interpersonal problems caused or exacerbated by the effects of the substance.

The term “substance dependence” as used herein, refers to a pattern of substance use, leading to clinically significant impairment or distress as manifested by at least three selected from the following group, occurring at any time within a period of time: (1) tolerance as defined by either (a) a need for substantially increased amounts of the substance to achieve the desired effect; or (b) substantially diminished effect with continued use of the same amount of the substance; (2) withdrawal, as demonstrated by either (a) the characteristic withdrawal syndrome for the specific substance; or (b) the same, or a closely related substance is taken to relieve or avoid withdrawal symptoms; (3) the substance is often taken in larger amounts or over a longer period then was intended; (4) there is a persistent desire or unsuccessful efforts to cut down or control substance use; (5) a great deal of time is spent in activities to obtain the substance, use the substance, or recover from its effects; (6) important social, occupational or recreational activities are given up or reduced because of substance use; and (7) the substance use is continued despite knowledge of having a persistent or recurrent physical or psychological problem that is likely to have been caused or exacerbated by the substance. Substance dependence can be with physiological dependence; that is evidence of tolerance or withdrawal is present, or without physiological dependence, where no evidence of tolerance or withdrawal is present.

The term “substance addiction” as used herein, refers to a chronic, relapsing disease characterized by a loss of control over drug use, compulsive drug seeking and craving for a substance, use that persists despite negative consequences, and physical and/or psychological dependence on the substance. Substance addiction typically follows a course of tolerance, withdrawal, compulsive drug taking behavior, drug seeking behavior, and relapse.

In some embodiments, the terms substance abuse, substance dependence, and substance addiction may be defined with reference to criteria set forth in the Diagnostic and Statistical Manual of Mental Disorders, 4th Ed. (1994) (“DSM-IV”).

The term “disease” refers to a state of health of a subject wherein the subject cannot maintain homeostasis, and wherein if the disease is not ameliorated then the subject's health continues to deteriorate. The term “disorder” refers to a state of health in which the subject is able to maintain homeostasis, but in which the subject's state of health is less favorable than it would be in the absence of the disorder. However, the definitions of “disease” and “disorder” as described above are not meant to supersede the definitions or common usage related to specific addictive diseases or disorders. For example, substance abuse related diseases and disorders, as used herein, include diseases and disorders related to substance abuse, substance dependence, and substance addiction.

The term “microbiota” is used interchangeably with “microbiome” and refers to the population of microorganisms present within or on a subject. The microbiota of a subject includes commensal microorganisms found in the absence of disease and may also include pathobionts and disease-causing microorganisms found in subjects with or without a disease or disorder. In some embodiments, the microbiota includes one or more bacterial communities that can be found or can exist (colonize) within a gastrointestinal tract of an organism. When referring to more than one microbiota, the microbiota may be of the same type (strain) or it may be a mixture of taxa, such as a mixture of Bacteroidetes, Firmicutes, Proteobacteria, Tenericutes, and Verrucomicrobia. In some aspects, the methods and compositions disclosed include altering the relative abundance of microbiota.

The term “fecal transplant” as used herein refers to fecal microbiota isolated from a healthy individual that does not have a substance-abuse related disease or disorder, which is transplanted into a recipient. In some embodiments, the fecal transplant is processed fecal material (fecal filtrate) having reduced volume and/or fecal aroma relative to unprocessed fecal material. In certain embodiments, the fecal transplant is a fecal bacterial sample. The term fecal transplant may also be used to refer to the process of transplantation of fecal bacteria isolated from a healthy individual into a recipient. The process may be also referred to as fecal microbiota transplantation (FMT), stool transplant or bacteriotherapy.

As used herein, the term healthy donor refers to individuals without a history of any chronic medical condition.

The term “therapeutically effective” refers to the amount of the composition used is of sufficient quantity to ameliorate one or more causes or symptoms of a disease or disorder. Such amelioration only requires a reduction or alteration, not necessarily elimination.

The term “pharmaceutically acceptable” refers to those compounds, materials, compositions, and/or dosage forms which are, within the scope of sound medical judgment, suitable for use in contact with the tissues of human beings and animals without excessive toxicity, irritation, allergic response, or other problems or complications commensurate with a reasonable benefit/risk ratio.

The term “carrier” means a compound, composition, substance, or structure that, when in combination with a compound or composition, aids or facilitates preparation, storage, administration, delivery, effectiveness, selectivity, or any other feature of the compound or composition for its intended use or purpose. For example, a carrier can be selected to minimize any degradation of the active ingredient and to minimize any adverse side effects in the subject.

The term “treatment” refers to the medical management of a patient with the intent to cure, ameliorate, stabilize, or prevent a disease, pathological condition, or disorder. This term includes active treatment, that is, treatment directed specifically toward the improvement of a disease, pathological condition, or disorder, and also includes causal treatment, that is, treatment directed toward removal of the cause of the associated disease, pathological condition, or disorder. In addition, this term includes palliative treatment, that is, treatment designed for the relief of symptoms rather than the curing of the disease, pathological condition, or disorder; preventative treatment, that is, treatment directed to minimizing or partially or completely inhibiting the development of the associated disease, pathological condition, or disorder; and supportive treatment, that is, treatment employed to supplement another specific therapy directed toward the improvement of the associated disease, pathological condition, or disorder.

The term “prevent” refers to a treatment that forestalls or slows the onset of a disease or condition or reduced the severity of the disease or condition. Thus, if a treatment can treat a disease in a subject having symptoms of the disease, it can also prevent that disease in a subject who has yet to suffer some or all of the symptoms.

The term “modulate(s)” as used herein includes inhibition, attenuation, control, diminishment, prevention, induction, detachment, removal, cleaning, and/or dispersal of microbial formation of growth, development, or behavior. The term “modulate” further refers to generating any change in the colonization or proliferation of a microbial population, to the induction of any change resulting in the increase or decrease of a physiological activity of a microbial population.

Reference will now be made in detail to specific aspects of the disclosed materials, compounds, compositions, articles, and methods, examples of which are illustrated in the accompanying Examples.

### Compositions

Disclosed herein are compositions that can treat a substance-abuse related disease or disorder in a subject. The compositions useful for treating the substance-abuse related disease or disorder can modulate the gut microbiota in the subject. In some embodiments, the compositions can increase or decrease the presence of one or more bacterial, viral, or eukaryotic species that reside in the gut.

In certain embodiments, the compositions for treating the subject with the substance-abuse related disease or disorder can alter the presence of one or more bacterial species of a genera of Akkermansia, Ruminococcus, Pseudobutyrivibrio, Coprococcus, Coprobacillus, Lactobacillus, Bifidobacteria, Verrucomicrobiae, Verrucomicrobia, Verrucomicrobiales, Clostridia, Bacilli, or Mollicutes. In certain embodiments, the composition can include an unknown and/or unculturable bacteria. For example, the composition can include an unknown, uncommon, and/or unculturable gut bacteria. In certain embodiments, the composition can alter the presence of one or more bacterial species of the Bacteriodes genus. In certain embodiments, the composition can alter the presence of one or more bacterial species of the Firmicutes genus.

In some examples, the composition can alter the presence of one or more bacterial species selected from Alistepes putredinis, Alistepes finegoldii, Anaerotruncus colihominis, Anaerofustis stercorihominis, Bacteroides thetaiotaomicron, Bacteroides vulgatus, Bacteroides ovatus, Bacteroides distasonis, Bacteroides salyersiae, Bacteroides stercoris, Bacteroides eggerthii, Bacteroides merdae, Bacteroides caccae, Bacteroides merdae, Bacteroides stercosis, Bacteroides uniformis, Bacteroides WH302, Bulleidia moorei, Bacteroides capillosus, Bifidobacterium animalis, Bifidobacterium breve, Bifidobacterium bifidum, Bifidobacterium longum, Bifidobacterium infantis, Bacillus coagulans, Clostridium leptum, Clostridium boltaea, Clostridium symbiosum, Clostridium scindens, Clostridium bartlettii, Clostridium spiroforme, Coprococcus catus, Catenibacterium mitsuokai, Coprococcus eutactus, Dorea formicigenerans, Dorea longicatena, Dialister sp., Eubacterium ventriosum, Eubacterium halii, Eubacterium siraeum, Eubacterium dolichum, Eubacterium cylindroides, or Eubacterium biforme, Eubacterium plautii, Faecalibacterium prausnitzii, Gemella haemolysans, Lactobacilllus lactis, Lactobacillus acidophilus, Lactobacillus bulgaricus, Lactobacillus casei, Lactobacillus fermentum, Lactobacillus gasseri, Lactobacillus plantarum, Lactobacillus reuteri, Lactobacillus rhamnosus (e.g., GG), Lactobacillus paracasei, Lactobacillus plantarus, Lactobacillus rhamnosus, Lactobacillus reuteri, Lactobacillus salivarius, Peptostreptococcus micros, Ruminococcus gnavus, Ruminococcus obeum, Ruminococcus torques, Ruminococcus callidus, Roseburia faecalis, Ruminococcus bromii, Subdoligranulum variabile, Saccharomyces boulardii, Streptococcus thermophiles, Streptoccocus salivarius K12, or Streptoccocus Salivarius M18.

In some embodiments, the composition can alter various combinations of microbiota species in the gut of the subject, such as at least two species, at least three species, at least four species, at least five species, at least six species, at least seven species, at least eight species, at least nine species, or at least ten species. In some embodiments, the composition can increase the presence of one or more bacterial, viral, or eukaryotic species that reside in the gut. In some embodiments, the composition can increase the presence of microbiota species that reside in the gut.

The composition can be in any suitable form. For example, the composition can be selected from a particular diet or diet ingredient, a probiotic, microbiota transplant, or combinations thereof. In some aspects, the composition can be a particular diet or diet ingredient that alters the amount of microbiota in the subject. In some embodiments, the diet ingredient can include a prebiotic. By way of example, one such diet ingredient is psyllium husks as described in U.S. Patent Application Publication No. 2006/0229905.

In some aspects, the composition for treating the subject with the substance-abuse related disease or disorder can be a microbiota transplant. In some embodiments, the microbiota transplant includes a fecal transplant. The fecal transplant can include fecal microbes isolated from a healthy individual, which is transplanted into a recipient. In certain embodiments, the fecal transplant is processed fecal material (fecal filtrate) having reduced volume and/or fecal aroma relative to unprocessed fecal material. In certain embodiments, the fecal transplant is a fecal bacterial sample.

It is not uncommon to find unknown bacteria, unculturable bacteria, or mixed cultures of bacteria in a fecal sample. In some embodiments, the composition can include one or more unknown and/or unculturable bacteria. The term “unculturable” as used herein refers to a given bacterium that current laboratory culturing techniques are unable to grow in the laboratory. An unculturable bacterium does not mean “a bacterium that can never be cultured” but, rather, signifies the lack of critical information on their biology. In some embodiments, the compositions described herein can include a substantially unculturable bacterium. The term “substantially unculturable” refers to a strain that, when cultured under normal laboratory conditions, less than 20% of replicates of that strain will reach a logarithmic growth phase, for example less than 20%, 15%, 10%, 5%, 2%, 1%, or 0.1%. Unknown and unculturable bacteria can be placed in taxonomic groups by amplifying their 16S rRNA gene, and subsequently their signature amplicon pattern can be recognized if they are encountered again.

In some aspects, the composition for treating the subject with the substance-abuse related disease or disorder can be a probiotic. The probiotic can include a therapeutically effective amount of fecal microbes and a pharmaceutically acceptable carrier. In some embodiments, the probiotic can include a carrier to facilitate the probiotics being delivered to the gastro-intestinal tract (e.g., the small intestine) in a viable and metabolically-active condition (see, e.g., Remington's Pharmaceutical Sciences, 16th Ed., Mac Publishing Company (1980). Suitable carriers for the present disclosure include those conventionally used, e.g., water, saline, aqueous dextrose, lactose, a buffered solution, starch, cellulose, glucose, lactose, sucrose, gelatin, malt, rice, flour, and the like. In some embodiments, the carrier provides a buffering activity to maintain the probiotic at a suitable pH to thereby exert a biological activity. In some embodiments, the microbiota can also be delivered in a condition capable of colonizing and/or metabolizing and/or proliferating in the gastrointestinal tract.

In some embodiments, the probiotic can include a coating resistant to gastric juice, thereby ensuring that the microbes contained in the composition can pass through the stomach unhindered and undamaged and the release of the microbiota first takes place in the upper intestinal regions. Standard encapsulation techniques known in the art can be used, and for example, as discussed in U.S. Pat. No. 6,190,591, which is hereby incorporated by reference in its entirety. Exemplary reagents for encapsulation include alginate. Other coating materials that can be used alone or combined with the alginate (or other encapsulating reagent) include whey protein, palm oil, xanthan gum, fat, cellulose acetate phthalate, polysaccharide chitosan, or starch. Other polysaccharides that have been used to encapsulate probiotics include xanthan gum, gum acacia, guar gum, locust bean gum, and carrageenan. Along with the protection that such coatings can offer to the microorganisms, other beneficial properties may also be imparted, such as giving greater control over release.

In some cases, the probiotic can be in suspension in a liquid that ensures physiological conditions for a probiotic microbiota. In some cases, the probiotic can be in a solid form, wherein the microbes can be present in free, preferably lyophilized form, or in immobilized form. For example, the microbes can be enclosed in a gel matrix which provides protection for the cells. In some examples, the probiotic can be a foodstuff. In this regard, the term “foodstuff” as used herein includes liquids (e.g., drinks), semi-solids (e.g., gels, jellies, yoghurt, etc.) and solids. Exemplary foodstuffs include dairy products, such as fermented milk products, unfermented mild products, yoghurt, frozen yoghurt, cheese, fermented cream, milk-based desserts milk powder, milk concentrate or cheese spread. Other products are also contemplated, such as soy-based products, oat-based products, infant formula, and toddler formula.

A variety of other compounds or agents can be useful for altering microbiota in a subject. Some of these compounds and agents are disclosed in U.S. Pat. No. 9,173,910 which is hereby incorporated by reference in its entirety. Such compounds or agents can include but are not limited to antibiotic treatments and/or antibacterial agents, prebiotics such as bacterial cell wall components, bacterial nucleic acids such as DNA and RNA, bacterial membrane components, and bacterial structural components such as proteins, carbohydrates, lipids and combinations of these such as lipoproteins, glycolipids and glycoproteins, organic acids, inorganic acids, bases, proteins and peptides, enzymes and co-enzymes, amino acids and nucleic acids, carbohydrates, lipids, glycoproteins, lipoproteins, glycolipids, vitamins, bioactive compounds, metabolites containing an inorganic component, small molecules, for example nitrous molecules or molecules containing a sulphurous acid, resistant starch, potato starch or high amylose starch, modified starches (including carboxylated starches, acetylated, propionated, and butyrated starches), non-digestible oligosaccharides such as fructooligosaccharides, glucooligosaccharides, xylooligosaccharides, galactooligosaccharides, arabinoxylans, arabinogalactans, galactomannans, polydextrose, oligofructose, inulin, derivatives of these, but not excluding other oligosaccharides able to exert prebiotic effects, other soluble fibers, and combinations thereof.

### Method

Methods for treating or preventing substance-abuse related diseases or disorders in a subject are disclosed. In some embodiments, the substance that is being abused can include an alcohol; an opioid; cocaine; marijuana; an amphetamine such as methamphetamine; a lysergic acid diethylamide (LSD); γ-hydroxybutanoic acid (GHB); a hallucinogen such as a ketamine, ayahuasca, dimethyltryptamine (DMT), mescaline, phencyclidine (PCP), and salvia; heroin; a steroid; a inhalant such as solvents, aerosols, and gases found in household products such as spray paints, markers, glues, and cleaning fluids; nitrites such as amyl nitrite; khat; kratom; ecstasy; over-the-counter cough/cold medicines; prescription sedatives; prescription stimulants; psilocybin; rohypnol; synthetic cannabinoids; synthetic cathinone; tobacco; and combinations thereof.

In some embodiments, the substance-abuse related disease or disorder is an alcohol-related disease or disorder. The alcohol-related disease or disorder that can be treated includes, but is not limited to, early-onset alcoholic, late-onset alcoholic, alcohol-induced psychotic disorder with delusions, alcohol addiction, alcohol abuse, excessive drinking, heavy drinking, problem drinking, alcohol intoxication, alcohol withdrawal, alcohol intoxication delirium, alcohol withdrawal delirium, alcohol-induced persisting dementia, alcohol-induced persisting amnestic disorder, alcohol dependence, alcohol-induced psychotic disorder with hallucinations, alcohol-induced mood disorder, alcohol-induced or associated bipolar disorder, alcohol-induced or associated posttraumatic stress disorder, alcohol-induced anxiety disorder, alcohol-induced sexual dysfunction, alcohol-induced sleep disorder, alcohol-induced or associated gambling disorder, alcohol-induced or associated sexual disorder, alcohol-related disorder not otherwise specified, alcohol intoxication, and alcohol withdrawal. In some examples, the alcohol-related disease or disorder is alcohol abuse, addiction, or dependency. In some embodiments, the subject with the alcohol-related disease or disorder may have decreased population of gut microbiota. In some embodiments, the subject with the alcohol-related disease or disorder may have decreased bacterial diversity of gut microbiota.

The method for treating or preventing substance-abuse related diseases or disorders can include modulating the gut microbiota in the subject with or susceptible to developing the disease or disorder. In some embodiments, the method for treating or preventing substance-abuse related diseases or disorders can include administering a composition as described herein. In some embodiments, the method for treating or preventing substance-abuse related diseases or disorders can include administering a microbiota transplant, a particular diet or diet ingredient, a probiotic, or a combination thereof to the subject.

The microbiota composition administered to the subject can be obtained from saliva, feces, and stomach, intestinal and/or rectal content; tissue sample from a digestive tract tissue such as an oral tissue, esophagus, stomach, intestine, ileum, cecum, colon and/or rectum; an ascites within a gastrointestinal tissue; and any other sample that may be used by those familiar with assessing microbiota. The identity and relative abundance of microbiota in a composition can be determined and/or measured. For example, the identification of the microbiota can be accomplished using culture-independent methods. For example, the microbiota can be identified by PCR using selective primers, quantitative PCR with selective primers, DNA-DNA hybridization, RNA-DNA hybridization and/or in situ hybridization. In some cases the hybridization is performed on a microarray. Additionally, PCR or high-throughput sequencing methods can detect over- and under-represented genes in the total bacterial population or transcriptomic or proteomic studies to identify lost or gained microbial transcripts or proteins within total bacterial populations. Alternatively, one or more species can be identified by determining the nucleotide sequence of a portion of a microbial genome, such as a 16S rRNA gene.

The methods can also include measuring total microbiota, individual microbiota taxa, such as phyla/classes/orders/families/genera/species, or measuring a combination of more than one microbiota taxa taken from a target location, or at a specific time before and/or after an activity, such as ingesting food or physical activity, or pre- or post-treatment. Individual relative abundances of microbiota may be obtained or total abundances of microbiota may be obtained over an extended time period. The abundances of microbiota can include one or more of any of the microbiota phyla/classes/orders/families/genera/species found in a gastrointestinal tract of an animal (e.g., a human), and can be performed by methods routinely used in the art including, gastrointestinal tract content sampling. The relative abundance or total abundances of microbiota may also include measuring total microbiota present in a sample.

The methods provided herein also can include methods of screening for and testing compositions, compounds, or agents for their ability to alter the relative abundance of select microbiota in a subject. Any of a variety of diagnostic factors can be monitored as indicators of efficacy, such as those known in the art. For example, weight changes, blood pressure, serum insulin/glucose levels, energy expenditure, breathing, color, temperature or other diagnostic indicators that can be measured to determine efficacy of the compound or agent. In addition, the presence or absence or level of one or more components in a sample from a subject can also be factors for determining efficacy of the compound or agent. Typical samples can include blood and urine samples, where the presence or absence or level of one or more components can be determined by performing, for example, a blood panel or a urine panel diagnostic test.

In some embodiments, the method of treating the subject includes administering a fecal transplant. In some embodiments, the method of treating the subject consists of administering a fecal transplant as described herein. The donor microbiota can be derived from a subject that is a rodent, a human, a livestock animal, a companion animal, or a zoological animal. In some embodiments, the donor microbiota is derived from a subject that is a human.

Fecal transplant has been used successfully for the treatment of Clostridium difficile infections (CDI), including therapy resistant forms thereof. As described in Borody et al. (Curr Gastroenterol Rep. 15: 337, 2013; the entire content of which is incorporated herein by reference) and understood in the art, a fecal transplant material is derived from healthy donors who have no risk factors for transmissible diseases and have not been exposed to agents, such as, for example, antibiotics, that could alter the composition of their gut microbiota. Fecal transplant donor selection criteria and screening tests are outlined in detail in published international guidelines established by the FMT Working Group (Bakken et al. Clin Gastroenterol Hepatol. 9:1044-9, 2011). Details pertaining to the harvesting and processing of fecal transplant material are known in the art and are reviewed in Borody et al. (supra). Briefly, many protocols call for use of fresh feces, which requires collection and processing on the same day scheduled for the FMT. Other protocols have been developed that use highly filtered human microbiota mixed with a cryoprotectant, which can be frozen for storage at −80° C. until required for use (Hamilton et al. Am J Gastroenterol. 107(5):761-7, 2012). This approach benefits from convenience with regard to scheduling, and generates a processed fecal material (fecal filtrate) having reduced volume and fecal aroma. Equivalent clinical efficacy can be expected when either purified processed fecal material or fresh, partly filtered feces are used in the disclosed methods. In some embodiments, the microorganism in the transplant can have undergone processing in order for it to increase its survival.

In some embodiments, the method of treating the subject can include administering a probiotic as described herein. In some embodiments, the method of treating the subject can include administering a particular diet or diet ingredient to modulate the gut microbiota in the subject. In some embodiments, the method of treating the subject can include administering one or more of a microbiota transplant, a probiotic, or a particular diet or diet ingredient. Administering a combination of more than one composition includes both simultaneous (at the same time) and consecutive administration in any order.

The dosage of the composition administered to the subject can vary depending on the particular composition, microbe(s) employed, and the effect to be achieved. For example, the dosage can contain a predetermined quantity of fecal microbiota calculated in an amount sufficient to treat the subject with the substance-abuse related disease or disorder. In some embodiments, the method can include administering the composition in an amount of about 10³ to about 10¹¹ CFU/g (colony forming units per gram) per dose. In some embodiments, the method can include administering the composition in an amount of about 10⁶ or greater, about 10⁷ or greater, about 10⁸ or greater, about 10⁹ or greater, or about 10¹⁰ or greater CFU organisms per dose. In some embodiments, the composition can be in a therapeutically effective amount to increase the amount of gastrointestinal microbiota when consumed by the subject.

The amount of microbiota, for example bacteria, administered to the subject in need of treatment can be determined according to various parameters such as the age, body weight, response of the subject, condition of the subject to be treated; the form of the composition in which the microbiota is included; the route of administration; and the required regimen. The severity of the condition may, for example, be evaluated, in part, by standard prognostic evaluation methods. For example, the amount of bacteria can be titrated to determine the therapeutically effective amount for administering to the subject in need of treatment. One of ordinary skill in the art would appreciate that the attending physician would know how to and when to terminate, interrupt or adjust administration of bacteria due to toxicity or organ dysfunctions. Conversely, the attending physician would also know to adjust treatment to higher levels if the clinical response were not adequate (precluding toxicity).

The amount of gut microbiota within the subject can be altered (i.e., increased or decreased) from about a one fold difference to about a ten fold difference or more, depending on the desired result and the individual subject. In certain embodiments, the abundance can be altered from about a two fold difference to about a ten fold difference, of about a three fold difference to about a ten fold difference, of about a four fold difference to about a ten fold difference, of about a five fold difference to about a ten fold difference, or of about a six fold difference to about a ten fold difference. Methods for determining the relative abundance of gut microbiota are known in the art. In some embodiments, the amount of gut microbiota within the subject can be increased from about a one fold difference to about a ten fold difference or more.

In some embodiments, the abundance of gut microbiota within the subject may be altered (i.e., increased or decreased) from about 1% to about 100% or more depending on the desired result and the individual subject. For example, the abundance may be altered by an increase of from about 20% to about 100%, from about 30% to about 100%, from about 40% to about 100%, from about 50% to about 100%, from about 60% to about 100%, from about 70% to about 100%, from about 80% to about 100%, or from about 90% to 100%. In some embodiments, the amount of gut microbiota within the subject can be increased from about 1% to about 100% or more.

The compositions described herein can be administered to the subject via various routes. For example, the composition can be administered to the subject via oral administration, rectal administration, transdermal administration, intranasal administration or inhalation. In some embodiments, the fecal transplant can be administered via naso-duodenal, transcolonoscopic, or enema based routes.

Administration of the composition can be intermittent, continuous, or chronic, as deemed appropriate by a practitioner, particularly in view of any change in the disease state or any undesirable side effects. “Chronic” administration refers to regular, long term administration which can be continuous or intermittent; “continuous” administration refers to treatment that is done without interruption; and “intermittent” administration refers to treatment that is done with interruption. In some embodiments, the disclosed composition can be administered on a daily basis or more or less often, depending on the survival of the microbiota in the subject. In some embodiments, the procedure of fecal transplantation can include single or multiple infusions (e.g., by enema) of fecal microbiota from a donor to the subject. In some embodiments, the composition can be administered with food or within three hours or two hours or one hour of consuming food. Consuming the composition with food or soon thereafter is likely to increase the survival of the microbiota by increasing the pH of the acidic components of the gastric or gastrointestinal tract.

The gut microbiota composition of the treated subject may be monitored before, during, or after the treatment period. A variety of monitoring techniques are known to one of ordinary skill in the art. For example, sequencing, PCR or microarray analysis may be used to identify the species and amount of bacteria present in the gut microbiota. ELISA assays using antibodies that specifically bind to bacterial antigens may also be used to identify and quantify the bacteria species in the gut microbiota. In some embodiments, administering the composition can also be adjusted according to the results from monitoring the composition of gut microbiota. For example, if the administered composition fully restores the normal microbiota colonization state of the subject, further administration of the composition may be suspended in view of further monitoring results.

The method of treating the subject can include administering a therapeutic agent in combination with the composition that modulates the gut microbiota. Suitable therapeutic agents include naltrexone (sold under the tradename Vivitrol™ and Revia™), disulfiram (sold under the tradename Antabuse™), or acamprosate (sold under the tradename Campral™).

In some embodiments, the compositions and methods described herein reduces the frequency of alcohol consumption compared with the frequency of alcohol consumption before the treatment. One of ordinary skill in the art will appreciate that the frequency can be compared with prior consumption by the subject or with consumption by a control subject not receiving the treatment. In some embodiments, the compositions and methods described herein reduces the quantity of alcohol consumed in a subject compared with the amount of alcohol consumed before the treatment or compared with the alcohol consumption by a control subject not receiving the treatment. In some embodiments, the compositions and methods described herein improves the physical or psychological sequelae associated with alcohol consumption compared with a control subject not receiving the treatment. In some embodiments, the compositions and methods described herein reduces increases the abstinence rate of a subject compared with a control subject not receiving the treatment. In some embodiments, the compositions and methods described herein reduces the average level of alcohol consumption in a subject compared with the level of alcohol consumption before the treatment or compared with the level of alcohol consumption by a control subject not receiving the treatment. In some embodiments, the compositions and methods described herein reduces alcohol consumption and increases abstinence compared with the alcohol consumption by the subject before treatment or with a control subject not receiving the treatment. In some embodiments, the compositions and methods described herein reduces treats a subject with a predisposition to alcohol abuse. In some embodiments, the compositions and methods described herein reduces treats a subject with a predisposition to alcohol dependence. In some embodiments, the compositions and methods described herein reduces treats a subject with a predisposition to alcohol addiction.

One of ordinary skill in the art will appreciate that there are multiple parameters or characteristics of alcohol consumption which may characterize a subject afflicted with an alcohol-related disease or disorder. It will also be appreciated that combination therapies can be effective in treating more than one parameter, and that there are multiple ways to analyze the effectiveness of treatment. The parameters analyzed when measuring alcohol consumption or frequency of alcohol consumption include, but are not limited to, heavy drinking days, number of heavy drinking days, average drinking days, number of drinks per day, days of abstinence, number of individuals not drinking heavily or abstinent over a given time period, and craving. Both subjective and objective measures can be used to analyze the effectiveness of treatment. For example, a subject can self-report according to guidelines and procedures established for such reporting. The procedures can be performed at various times before, during, and after treatment. Additionally, assays are available for measuring alcohol consumption.

1. A method of treating a subject for substance-abuse related disease or disorder, comprising administering to the subject a microbiota transplant.

2. The method of claim 1, further comprising administering a probiotic, therapeutic agent, or a combination thereof to the subject.

3. The method of claim 1, wherein the method includes administering a composition comprising at least one bacterial species of a genera of Akkermansia, Ruminococcus, Pseudobutyrivibrio, Coprococcus, Coprobacillus, Lactobacillus, Bifidobacteria, Clostridia, Verrucomicrobiae, Verrucomicrobia, or Verrucomicrobiales.

4. The method of claim 1, wherein the method includes administering an unculturable bacteria.

5. The method of claim 1, wherein the microbiota transplant is a fecal transplant.

6. The method of claim 1, wherein the fecal transplant is administered in a therapeutically effective amount to increase the amount of gastrointestinal microbiota when consumed by the subject.

7. The method of claim 1, wherein the fecal transplant comprises from about 10³ to about 10¹¹ colony forming units per gram.

8. The method of claim 1, wherein the fecal transplant is administered orally or anally.

9. The method of claim 1, wherein the composition is administered intermittently, periodically, continuously, or chronically.

10. (canceled)

11. (canceled)

12. The method of claim 1, wherein the composition is administered for up to three weeks.

13. (canceled)

14. (canceled)

15. The method of claim 1, further comprising administering a therapeutic agent in combination with the composition that modulates the gut microbiota.

16. (canceled)

17. The method of claim 1, wherein the substance-abuse related disease or disorder is an alcohol-related disease or disorder.

18. The method of claim 1, wherein the substance-abuse related disease or disorder is an opioid-related disease or disorder.

19. (canceled)

20. (canceled)

21. (canceled)

22. A method for treating a subject for alcohol abuse, addiction, or dependency, the method comprising administering to the subject a therapeutically effective amount of a composition comprising a fecal transplant isolated from a healthy donor.

23. A method for treating a subject for alcohol abuse, addiction, or dependency, the method comprising controlling the microbial population in the subject's gut.

24. The method of claim 23, comprising administering to the subject a therapeutically effective amount of a composition comprising a microbiota transplant.

25. (canceled)

26. (canceled)

27. (canceled)

28. The method of claim 24, wherein the microbiota transplant is a fecal transplant.

29. (canceled)

30. (canceled)

31. (canceled)

32. (canceled)

33. (canceled)

34. (canceled)

35. (canceled)

36. (canceled)

37. (canceled)

38. (canceled)

39. (canceled)

40. The method of claim 22, wherein the composition is administered in the upper or lower portion of a human or animal gastrointestinal tract.

41. (canceled)

42. The method of claim 22, wherein the substance abuse, addiction, or dependency is alcohol addiction.

43. (canceled)

44. (canceled)

45. (canceled)

46. The method of claim 22, wherein the substance-abuse related disease or disorder is an opioid-related disease or disorder.

47. (canceled)

48. (canceled)

49. (canceled)
